(19) United States
(12) Patent Application Publication
(10) Pub. No.: US 2026/0069773 A1
(43) Pub. Date: Mar. 12, 2026

(54) NEEDLE, NEEDLE INSERTER ASSEMBLY AND METHOD OF INSERTING A NEEDLE

(71) Applicant: Unomedical A/S, Lejre (DK)

(72) Inventors: Erik Othel-Jacobsen, Snekkersten (DK); Henrik Tipsmark, Roskilde (DK); Rune Frimand Byager, Hvalsoe (DK)

(21) Appl. No.: 19/393,897

(22) Filed: Nov. 19, 2025

Related U.S. Application Data
(63) Continuation of application No. PCT/EP2024/063733, filed on May 17, 2024.
(60) Provisional application No. 63/503,272, filed on May 19, 2023.
(60) Provisional application No. 63/503,315, filed on May 19, 2023.

(30) Foreign Application Priority Data
Jul. 6, 2023 (GB) 2310430.0
Jul. 6, 2023 (GB) 2310431.8

(51) Int. Cl. A61M 5/158

(52) U.S. Cl. CPC A61M 5/158; A61M 2005/1585; A61M 2005/1586; A61M 2205/0216; A61M 2205/0266

(57) ABSTRACT

A needle inserter assembly, particularly an assembly including a needle mounted to a support member for engaging a base member. The assembly includes a support member including a body portion and a needle, wherein the needle includes a lumen, a distal portion having a tip configured to facilitate insertion into a soft tissue of a patient, and a proximal portion mounted to the body portion so that the proximal portion extends along a longitudinal axis of the body portion. The assembly also includes a base member including a receiving portion configured to receivingly engaging the support member as the support member is moveable from a first position, in which the tip engages a first guide element within the base member, to a second position in which the tip extends out of an opening provided through the base member.

### TECHNICAL FIELD

The present invention relates to needle inserter assembly, particularly an assembly including a needle mounted to a support member for engaging a base member. The invention also relates to a method of inserting a needle, particularly a method having a lumen for distributing fluid into, or removing fluid from a soft tissue of a patient.

### BACKGROUND

Needles are a typical feature of a wide variety of medical devices, and are commonly used in many percutaneous medical procedures, including injection, infusion and blood sampling. Insertion of a needle into soft tissue is a process involving cutting of the tissue, and is dependent upon the cutting edge geometry of the tip of the needle. The geometry is designed to cut through the tissue effectively to minimise insertion forces, and thereby minimise tissue deformation and needle deflection during insertion. It is generally recognised that minimising tissue deformation eases pain and tissue trauma associated with insertion, and minimising needle deflection improves placement of the needle.

During insertion of needles, an arcuate cut is made in the tissue, extending from each side of the needle point as the needle advances. As the cut continues to extend, a hinged flap of tissue overlaying the lumen of the needle is formed. A cutting edge of the needle further extends the cut such that the flap is severed, and subsequently the flap may be pushed into the lumen by further advancement of the needle. This undesirable mechanism is known as coring. Coring may similarly occur when a needle is inserted through a pharmaceutical closure, i.e. a bung or plug, of a container of medicament to draw up medicament therefrom, wherein a fragment of the material forming the closure is cut away. Coring is undesirable as it reduces the efficacy of the needle, and consequently the efficacy of delivery of medicament to a patient. Coring may lead to contamination of medicament and/or injection of a foreign body, for example a severed fragment of the material forming pharmaceutical closure, into a patient. Coring may increase foreign body response (FBR) to the medical procedure in question.

Needle inserter assemblies and mechanisms are frequently used to facilitate insertion of a needle and/or cannula into soft tissue of a patient to further distribute fluid into, or remove fluid from, the body of the patient. Typically, the needle is are positioned within the inserter mechanism in a retracted position and has a substantially linear dimension. The needle inserter assembly is then activated so that the needle is urged axially along a longitudinal axis of the needle to extend from the retracted position into the soft tissue of the user. The needle inserter assembly may be required to remain mounted to the soft tissue for a several days or more so that the inserted needle or cannula can continuously distribute or remove fluid from the soft tissue over a prolonged period.

Typically, the needle are positioned within the inserter mechanism in a retracted position and has a substantially linear insertion path. The inserter mechanism may be activated wherein the needle is extended by the mechanism axially along a longitudinal axis of the needle into soft tissue of the user. The inserted needle or cannula typically remains in the patient for a period o time. Accordingly, the bulky inserter mechanism is often removed after the needle and/or cannula is inserted to reduce the size and weight of the device while in use. After the needle and/or cannula is inserted into the user, it is often fluidly coupled to a fluid delivery or retention device.

It would be useful to provide a needle inserter assembly, as well as a method of inserting a needle into the soft tissue of a patent, to reliably insert a needle at an optimal angle to the soft tissue. That is, it would be useful to insert a needle with a reliably controlled insertion axis.

In particular, it would be useful to enable a needle to be inserted in a way that reduces or minimises lateral forces on the needle as it is inserted. In other words, it would be useful to insert a needle so that the urging force applied to the needle to insert the needle into the soft tissue acts along the needle insertion axis.

It would also be useful to simplify the operation of needle inserter assembly. It would be useful to insert a needle so that it maintains a reliable flow of fluid through the lumen.

It would be useful to provide convenient insertion of a needle into soft tissue, particularly so that the needle is securely mounted to the soft tissue for use over a prolonged period, such as several days or more.

### BRIEF SUMMARY

The invention is set out in the appended claims.

According to one aspect, there is provided a needle inserter assembly including:
a support member including a body portion and a needle, wherein the needle includes a lumen, a distal portion having a tip configured to facilitate insertion into a soft tissue of a patient, and a proximal portion mounted to the body portion so that the proximal portion extends along a longitudinal axis of the body portion, and
a base member including a receiving portion configured to receivingly engage the support member as the support member is moveable from a first position, in which the tip engages a first guide element within the base member, to a second position in which the tip extends out of an opening provided through the base member,
wherein, in the second position, the support member is mounted to the base member with the longitudinal axis oriented in a first direction, and
wherein the first guide element is configured so that, as the support member moves from the first position towards the second position, the first guide element contactingly engages the needle to guide the tip towards the opening in a second direction, wherein the second direction is at an angle to the first direction.

Suitably, the base member may include a receiving portion configured to receivingly engage the support member with the longitudinal axis oriented in a second direction as the support member is moved from the first position towards the second position. That is, the longitudinal axis of the body portion may be oriented in the second direction, at an angle to the first direction, by the receiving portion as the support member is moved from the first position towards the second position.

Suitably, the support member is releasably mounted to the base member. That is, the support member is separable from the base member prior to assembly for use, or when the needle is withdrawn from soft tissue after use.

Suitably, as the support member moves from the first position to the second position, the first guide element may contactingly engage an intermediate portion of the needle.

Suitably, the support member may be substantially planar along the longitudinal axis. The longitudinal axis may be a central longitudinal axis of the support member.

Suitably, the support member may include a port and the proximal portion is fluidly connected to the port.

Suitably, as the support member moves from the first position to the second position, the first guide element may guide the tip at an angle to the longitudinal axis of the support member.

Suitably, the latch element may include at least one tang extending from the body portion in the lengthwise direction, wherein each tang of the at least one tang is configured to be resiliently deflectable as the support member moves towards the second position.

Suitably, a tang may be mounted to a projecting element.

Suitably, the support member may include a latch element configured to lock, preferably releasably lock, the support member to the base member in the second position.

Suitably, the support member may include at least one projecting element mounted to the body portion to extend away from the body portion and wherein the projecting element is configured to contactingly engage the receiving portion of the base member as the support member moves from the first position to the second position.

Suitably, the base member may include a bore extending through the base member to the opening, and wherein first guide element includes an inner surface of the bore.

Suitably, the bore may extend to the opening along an insertion axis and wherein the insertion axis is oriented in the second direction.

Suitably, in the second position the tip may extend out of the opening in the second direction, and wherein the second direction is disposed at an angle relative to the engaging surface in a range of from 20° to 40°, preferably in a range 25° to 35°.

Suitably, in the first position, the distal portion, the intermediate portion, and the proximal portion may be aligned along the longitudinal axis of the support member.

Suitably, the base member may include an engaging surface configured to contact the soft tissue of a patient during insertion of the tip of the needle into the soft tissue, and wherein the opening is located in the engaging surface.

Suitably, in the second position, the distal portion may be disposed in the second direction and the proximal portion is disposed in the first direction.

Suitably, in the first position, the distal portion and the proximal portion may be aligned along the longitudinal axis of the support member.

Suitably, the needle may include an intermediate portion and, in the second position, the intermediate portion is deformed by the first guide element.

Suitably, the receiving portion may include at least one recessed cavity for receiving at least a portion of the support member.

Suitably, each recessed cavity may extend inwardly from the receiving portion in the first direction.

Suitably, the needle may be elastically deformable.

Suitably, the needle may be formed from a pseudo-elastic material or a superelastic material.

Suitably, the needle may be formed from a nitinol material, typically a nitinol material with an austenite finish temperature in a range of from −15° C. to 30° C., preferably in a range of from 0° C. to 20° C.

Suitably, the needle may have a wall thickness of defined between an outer surface and a lumen surface, and wherein the wall thickness is at least 20 μm. Suitably, at least a portion of the needle may have a wall thickness of defined between a portion of the outer surface and a portion of the lumen surface, wherein the wall thickness is at least 20 μm. One or more of the distal portion, the intermediate portion, or the proximal portion of the needle may have a wall thickness of defined between respective portions of an outer surface and a lumen surface, wherein the wall thickness is at least 20 μm. More suitably, a wall thickness may be less than 400 μm.

Suitably, the needle may comprise an outer diameter, wherein the outer diameter is less than 0.7 mm. Suitably, at least a portion of the needle may have an outer diameter of less than 0.7 mm. One or more of the distal portion, the intermediate portion, or the proximal portion of the needle may have an outer diameter of less than 0.7 mm. More suitably, an outer diameter may be at least 0.4 mm.

Suitably, the needle may comprise an inner diameter, wherein the inner diameter is at least 0.2 mm. Suitably, at least a portion of the needle may have an inner diameter of at least 0.2 mm. One or more of the distal portion, the intermediate portion, or the proximal portion of the needle may have an inner diameter of at least 0.2 mm. More suitably, an inner diameter may be less than 0.5 mm.

According to a further aspect, there is provided a needle inserter assembly including:
a support member including a body portion and a needle, wherein the needle includes a lumen, a proximal portion mounted to the body portion, and a distal portion having a tip configured to facilitate insertion into a soft tissue of a patient, and
a base member includes a receiving portion configured to receivingly engaging the support member as the support member is moveable from a first position, in which the tip is retracted within the base member to a second position in which the tip extends out of an opening provided through the base,
wherein the base member includes a first guide element, and
wherein the receiving portion is configured so that, as the support member moves from the first position towards the second position, the first guide element contactingly engages the needle to guide the tip in a first direction towards the opening, and the receiving portion contactingly engages the support member to deflect the body portion at an angle to the first direction.

According to yet another aspect, there is provided a method of inserting a needle having a lumen for distributing fluid into, or removing fluid from a soft tissue of a patient, the method including:
providing a support member of a needle inserter assembly, wherein the support member includes a body portion and a needle, and wherein the needle includes a proximal portion mounted to the body portion, and a distal portion having a tip configured to facilitate insertion into a soft tissue of a patient,
engaging a base member of the needle inserter assembly with the soft tissue, wherein the base member includes a receiving portion configured to receivingly engaging the support member, an opening, and a first guide element,
engaging the support member with the receiving portion of the base member in a first position so that the needle engages the first guide element with the distal portion oriented in a second direction and with the tip of the needle retracted within the base member,
moving the support member in a first direction to a second position, wherein the first direction is at an angle to the second direction, and
guiding the tip of the distal portion in the second direction with the first guide element as the support member is moved towards the second position.

Suitably, the step of guiding the tip of the distal portion may move the tip towards and through the opening in the base member.

Suitably, the step of moving the support member from the first position to the second position may elastically deforms the needle.

Suitably, the first guide element may engage an intermediate portion of the needle. Other technical features may be readily apparent to one skilled in the art from the following figures, descriptions, and claims.

Suitably, the base member, and the needle may be coupled to a patient via an adhesive patch or the like. The adhesive patch may be mounted to a lower surface of the base member.

Suitably, the needle inserter assembly may be at least partially enclosed by a protective cover, such as a wound dressing cover, when coupled to the patient.

Suitably, a protective cap may be mounted to the support member prior to use. The protective cap may include an elongate bore to enclose the needle when the protective cap is mounted to the support member.

According to an aspect, a needle inserter assembly includes a base member, and a needle, the needle includes a lumen, a proximal portion, and a distal portion having a tip configured to facilitate insertion into a soft tissue of a patient, where the needle is operably mountable within the needle inserter assembly so that the distal portion is moveable from a first position, in which the distal portion projects through an opening provided through the base member, to a second position in which the distal portion is retracted within the base member, and where the needle is elastically deformable to provide an urging force to move the distal portion back towards the first position when the distal portion is moved towards the second position.

In this way, by moving the distal portion towards the second position, the needle is moved from an un-deformed state to an energised state. Release of the needle from an energised state causes the needle to recover so that the needle itself provides the urging force to move the distal portion back towards the first position.

Suitably, the needle may include an intermediate portion disposed between the proximal portion and the distal portion.

Suitably, the proximal portion may be fixedly mountable within the needle inserter assembly.

Suitably, in the first position, the proximal portion and the distal portion may be aligned along a needle axis.

Suitably, in the second position, an intermediate portion of the needle may be displaced away from the needle axis.

Suitably, the urging force may be provided in a direction along the needle at the distal portion.

Suitably, the needle inserter assembly may include a first engaging element configured to contactingly engage the needle and displace the intermediate portion of the needle to thereby move the distal portion from the first position to the second position.

Suitably, the first engaging element may be rotatably mounted within the needle inserter assembly and configured to rotate around an element axis disposed at an angle to a needle axis. Preferably the element axis disposed perpendicular to the needle axis.

Suitably, the first engaging element may be rotatably mounted within the needle inserter assembly and configured to both rotate around an element axis disposed at an angle to a needle axis as well as translate along the element axis during rotation. For example, the first engaging element may be mounted on a screw thread within the needle inserter assembly. Preferably the element axis is disposed perpendicular to the needle axis. In this way, an initial rotation of the first engaging element provides contacting engagement to displace the intermediate portion of the needle. Then, further rotation translates the first engaging element along the element axis to release the intermediate portion. The further rotation may cause the first engaging element to be translated along the element axis either towards or away from the cover of the needle inserter assembly.

Suitably, the first engaging element may include an actuator configured to be operably engaged by a user to manually rotate the first engaging element around the element axis.

Suitably, the needle inserter assembly may include a second engaging element configured to contactingly engage the needle to selectively displace the intermediate portion towards the needle axis and provide a secondary urging force to move the distal portion at least partially back towards the first position.

Suitably, the base member may include an engaging surface configured to contact the soft tissue of a patient during insertion of the tip of the needle into the soft tissue, and where the opening is located in the engaging surface.

Suitably, in the first position, at least the tip of the distal portion extends out of the opening along an insertion axis, wherein the insertion axis is disposed relative to the engaging surface at an angle in the range of from 10° to 80°, preferably 20° to 60°, more preferably 30° to 45°.

Suitably, the base member may include a bore extending from an upper surface of the base member to the opening, wherein the distal portion may be reciprocally moveable within the bore.

Suitably, the bore may be configured to constrain the distal portion to move along an insertion axis when moving between the first position and the second position.

Suitably, the bore may extend from the upper surface to the opening along an insertion axis, and where the insertion axis is disposed relative to the engaging surface at an angle in a range of from 10° to 80°, preferably from 20° to 60°, more preferably from 30° to 45°.

Suitably, the needle inserter assembly may include a port configured to receive the proximal portion of the needle and fluidly connect the needle to a fluid channel to distribute fluid into, or remove fluid from, the soft tissue of a patient.

Suitably, the urging force provided to the distal portion may be a maximal urging force when the distal portion is in the second position.

Suitably, the urging force provided to the distal portion when the distal portion is in the second position may be in a range of from 1N to 5N, preferably from 2N to 4N, more preferably from 2.5N to 3N.

Suitably, the needle may formed from a pseudo-elastic material or a superelastic material.

Suitably, the needle may be formed from a nitinol material, typically a nitinol material with an austenite finish temperature in the range of from −15° C. to 30° C., preferably in the range of from 0° C. to 20° C.

Suitably, the needle may be elastically deformable in a range of from greater than 0% to 9%, preferably a range of from greater than 0% to 6%.

Suitably, the needle may have a wall thickness of defined between an outer surface and a lumen surface, and wherein the wall thickness is at least 20 μm. Suitably, at least a portion of the needle may have a wall thickness of defined between a portion of the outer surface and a portion of the lumen surface, wherein the wall thickness is at least 20 μm. One or more of the distal portion, the intermediate portion, or the proximal portion of the needle may have a wall thickness of defined between respective portions of an outer surface and a lumen surface, wherein the wall thickness is at least 20 μm. More suitably, a wall thickness may be less than 400 μm.

Suitably, the needle may comprise an outer diameter, wherein the outer diameter is less than 0.7 mm. Suitably, at least a portion of the needle may have an outer diameter of less than 0.7 mm. One or more of the distal portion, the intermediate portion, or the proximal portion of the needle may have an outer diameter of less than 0.7 mm. More suitably, an outer diameter may be at least 0.4 mm.

Suitably, the needle may comprise an inner diameter, wherein the inner diameter is at least 0.2 mm. Suitably, at least a portion of the needle may have an inner diameter of at least 0.2 mm. One or more of the distal portion, the intermediate portion, or the proximal portion of the needle may have an inner diameter of at least 0.2 mm. More suitably, an inner diameter may be less than 0.5 mm.

Suitably, the needle may include an intermediate portion, wherein the intermediate portion is deflectable around an arcuate path with a pitch radius of at least 3.5 mm. Suitably, the pitch radius may be in a range for from 3.5 mm to 20 mm, preferably in a range of from 4 mm to 12 mm.

In another aspect, a method of inserting a needle having a lumen for distributing fluid into, or removing fluid from a soft tissue of a patient is provided. The method includes: providing a needle, wherein the needle includes a proximal portion and a distal portion having a tip configured to facilitate insertion into a soft tissue of a patient; providing a base member with needle operably mounted therein; elastically deforming the needle to moving the distal portion to a second position, in which the distal portion is retracted within the base member; engaging the base member on the soft tissue; and releasing the distal portion from the second position so that the needle provides an urging force to move the distal portion back towards a first position in which the distal portion extends from the base member.

Suitably, the step of elastically deforming the needle may provide an elastic deformation of an intermediate portion of the needle in a range of from greater than 0% to 9%, preferably a range of from greater than 0% to 6%.

Suitably, the method may include a further step of providing a secondary urging force to move the distal portion at least partially back towards the first position.

Suitably, the method may include a further step of constraining the distal portion and the proximal portion along a needle axis as the needle is moves between the first position and the second position.

Other technical features may be readily apparent to one skilled in the art from the following figures, descriptions, and claims.

Certain examples provide a convenient insertion of a needle into soft tissue, in particular, along a controlled insertion axis. That is, needle insertion into soft tissue is provided conveniently at a predetermined insertion angle relative to the soft tissue.

Certain examples ensure that the needle is urged into the soft tissue without lateral deflection. In particular, an urging force used to incise the soft tissue with the sharpened tip, is provided directly along the lengthwise axis of the distal portion of the needle.

Certain examples allows for orientations in which the needle is not aligned ideally with the insertion axis.

Certain examples ensure the tip of the needle is inserted into the soft tissue along a predetermined insertion axis, independent from the means of locking engagement.

Certain examples ensure the tip of the needle is secured within the needle inserter assembly so that the needle inserter assembly is convenient to use in a space-efficient manner.

Certain examples provide a needle inserter assembly or a method that ensures the needle is inserted easily. In particular, the tip is urged to pierce the soft tissue with a consistent urging force. The tip is urged to pierce the soft tissue without requiring manual manipulation or orientation, improving reliability and repeatability of the insertion method.

Certain examples provide a convenient insertion axis of a needle into soft tissue, in particular, along a controlled insertion axis, that is at a controlled insertion angle relative to the soft tissue.

Certain examples ensure the needle is urged into the soft tissue without lateral deflection.

Certain examples ensure the tip of the needle is inserted into the soft tissue along a predetermined insertion axis.

### BRIEF DESCRIPTION OF THE SEVERAL VIEWS OF THE DRAWINGS

Examples of the invention are now described, by way of example only, hereinafter with reference to the accompanying drawings, in which:

FIG. 1 shows an expanded perspective view of an example needle inserter assembly;

FIG. 2 shows an upper view of the support member of the example of FIG. 1 with fluid delivery components mounted thereto;

FIG. 3 shows an upper view of the base member of the example of FIG. 1;

FIG. 4 shows a rear perspective view of the base member of the example of FIG. 1;

FIG. 5 shows a perspective view of the support member of FIG. 1 oriented to be engaged with the respective base member;

FIG. 6 shows a perspective view of the support member of FIG. 1 mounted to the respective base member in a second position;

FIG. 7 shows an upper cross-sectional view of the mounted configuration of FIG. 6;

FIG. 8 shows a perspective view of the example of FIG. 6 for use with a patient;

FIG. 9A to FIG. 9C show side cross-sectional perspective views of the support member of FIG. 1 as it is moved relative to the respective base member into the second position;

FIG. 10 shows a graph illustrating certain properties of a nitinol material;

FIG. 11 shows a stress-strain diagram comparing certain properties of a needle formed from nitinol material with those of a needle formed of stainless steel;

FIG. 12 shows a graph illustrating certain properties of needles formed from a nitinol material;

FIG. 13 shows an upper perspective view of an example needle inserter assembly;

FIG. 14 shows a lower perspective view of the example of FIG. 13, with the distal portion in the second position;

FIG. 15 shows a cross-sectional perspective view of the example of FIG. 13, with the distal portion in the first position; and

FIG. 16 shows a magnified view of a portion of the example of FIG. 15.

### DETAILED DESCRIPTION

Certain terminology is used in the following description for convenience only and is not limiting. The words ‘right’, ‘left’, ‘lower’, ‘upper’, ‘front’, ‘rear’, ‘upward’, ‘down’ and ‘downward’ designate directions in the drawings to which reference is made and are with respect to the described component when assembled and mounted. The words ‘inner’, ‘inwardly’ and ‘outer’, ‘outwardly’ refer to directions toward and away from, respectively, a designated centreline or a geometric centre of an element being described (e.g. central axis), the particular meaning being readily apparent from the context of the description.

Further, as used herein, the terms ‘connected’, ‘attached’, ‘coupled’, ‘mounted’ are intended to include direct connections between two members without any other members interposed therebetween, as well as, indirect connections between members in which one or more other members are interposed therebetween. The terminology includes the words specifically mentioned above, derivatives thereof, and words of similar import.

Further, unless otherwise specified, the use of ordinal adjectives, such as, “first”, “second”, “third” etc. merely indicate that different instances of like objects are being referred to and are not intended to imply that the objects so described must be in a given sequence, either temporally, spatially, in ranking or in any other manner.

As used herein, elastic deformation refers to the recoverable deformation of at least a portion of a needle so that the portion returns from a deformed, energised state to an un-deformed state.

As used herein, the needle axis is the central longitudinal axis of the needle.

In certain examples, an elastic deformation gives a lengthwise extension, or a lengthwise compression, of a portion of the needle along a linear axis, particularly along the needle axis. That is, in the un-deformed state, the portion of the needle is disposed along a needle axis and, in the energised state, the portion of needle is elastically extended or compressed along the needle axis.

In certain examples, an elastic deformation gives a deformation of a portion of the needle to curve the needle. That is, in the un-deformed state, the portion of the needle lies along a needle axis and, in the energised state, the portion of the needle is deformed away from the needle axis. In this way, a region of the needle on an outside of the curve caused by deforming the needle is extended elastically and another region of the needle on an inside of the curve caused by deforming the needle is compressed elastically.

As used herein, a deformation limit refers to a limit of deformation of at least a portion of the needle when it is elastically deformed lengthwise along a linear axis and/or a limit of deformation of a portion of a needle that is elastically deformed to form a curve. The deformation limit is thereby the limit of any localised elastic extension or localised elastic compression within the region of the curved portion of the needle.

Certain examples described here may have particular application for use in medical devices for continuous subcutaneous insulin infusion (CSII) therapy, for example infusion sets. However, other applications are contemplated, such as cannula access ports and infusion pump systems, including patch pumps and so-called closed-loops systems, which are infusion pump systems used in combination with a continuous glucose monitoring (CGM) device to continually monitor blood sugar levels and adjust the amount of insulin delivered to a patient automatically. Certain examples have particular application for use in needles formed of a superelastic material, for example a nickel titanium alloy, i.e. Nitinol.

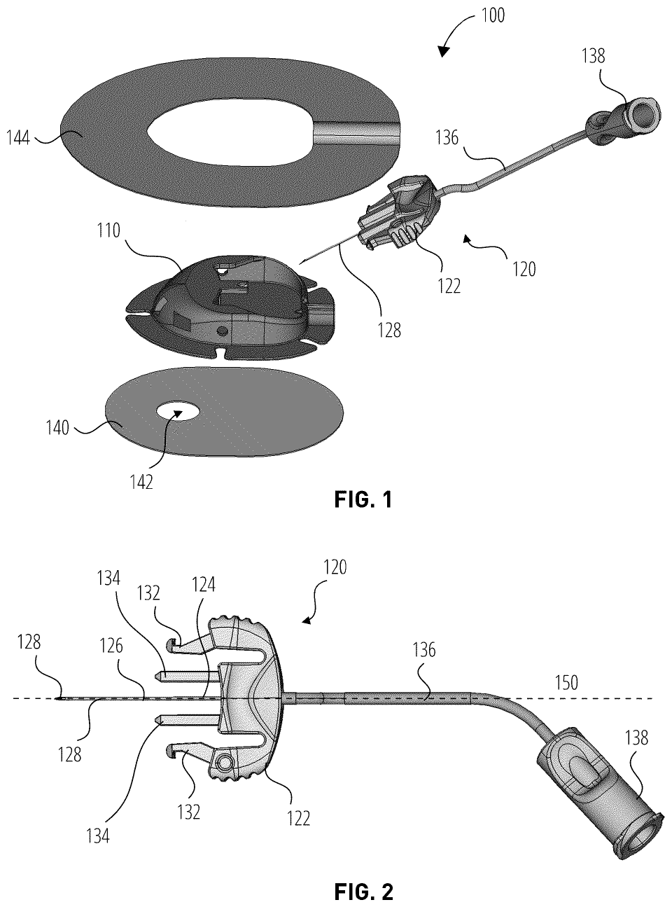

Referring now to FIG. 1 to FIG. 4, there is shown an expanded view of an example needle inserter assembly 100 according to an aspect. The needle inserter assembly 100 includes a base member 110 and a support member 120 mountable to the base member 110.

The support member 120 includes a body portion 122 and a needle, wherein the needle includes a lumen, a distal portion 128 having a tip configured to facilitate insertion into a soft tissue of a patient, and a proximal portion 124 mounted to the body portion 122 so that the proximal portion 124 extends along a longitudinal axis 150 of the body portion 122.

The base member 110 includes a receiving portion 106 configured to receivingly engaging the support member 120 as the support member 120 is moved from a first position, in which the tip engages a first guide element 112 within the base member 110, to a second position in which the tip extends out of an opening 114 provided through the base member 110.

The needle inserter assembly 100 includes an adhesive patch 140. The adhesive patch 140 is suitably tacky to affix a lower surface of the base member 110 to the soft tissue of the patient. With the needle inserter assembly 100 affixed to the patient, the needle of the support member 120 may be mounted to the needle inserter assembly 100, as described herein, so that the needle is inserted into the soft tissue of the patient. As will appreciated, the needle inserter assembly 100 may be affixed or otherwise secured to the patient without an adhesive patch, instead using other suitable means.

The adhesive patch 140 includes an aperture 142 extending therethrough. The adhesive patch 140 is suitably positioned to align with the opening 114 on the lower surface of the base member 110 when the adhesive patch 140 is fixed to the lower surface.

The needle inserter assembly 100 also includes a protective cover 144 mountable to an upper surface of the base member 110. The protective cover 144 may be mounted to the base member 110 after the support member 120 is engaged with the base member 110 in the second, enclosing an outer perimeter of the base member 110 against the soft tissue of the patient. The protective cover 144 thereby reduces ingress of dirt or contamination between the soft tissue and the needle inserter assembly 100 during use.

The support member 120 includes a tubing 136 mounted to the support member 120 and fluidly connected to the lumen of the distal portion 128. The proximal portion 124 is fluidly connected to the lumen via a port in the support member 120.

A connector 138 is fluidly connected to the tubing 136 at an end portion of the tubing 136 distal to the body portion 122.

Referring specifically to FIG. 2, there is shown the support member 120 suitable for mounting to the base member 110 of the needle inserter assembly 100. A tubing 136 and connector 138 is shown coupled to the body portion 122 of the base member 110 and arranged to be fluidly connected to the needle via a port in the body portion 122. The tubing 136 and connector 138 each include a respective lumen to for delivery or removal of fluid to the needle of the support member 120.

The support member 120 is substantially planar along the longitudinal axis 150. That is, the support member 120 extends in a longitudinal axis 150 and in a transverse axis extending perpendicular to the longitudinal axis 150. The body portion 122 and distal portion 128 of the needle are spaced apart along the longitudinal axis 150. A first side and a second side of the body portion 122 are spaced apart along the transverse axis.

In the example, the longitudinal axis 150 is a central longitudinal axis of the support member 120. In the example, the transverse axis is a central transverse axis of the support member 120.

The body portion 122 further includes an upper surface and an opposing, lower surface oriented to face away from both the longitudinal axis 150 and the transverse axis. The upper surface is spaced apart from the lower surface to provide a thickness dimension to the body portion 122 of the support member 120.

The support member 120 includes a port, particularly in the present example the body portion 122 of the support member 120 includes a port. The proximal portion 124 of the needle is fluidly connected to the port. In this way, the lumen of the needle is fluidly connected to the tubing 136 and connector 138 for delivery of fluid to the needle, or removal of fluid from the needle.

The needle includes an intermediate portion 126 disposed between the distal portion 128 of the needle and the proximal portion 124 of the needle. In the first position, the distal portion 128, the intermediate portion 126, and the proximal portion 124 are aligned along the longitudinal axis 150 of the support member 120.

The needle is elastically deformable. That is, the needle is configured to give a recoverable deformation when used in the example needle inserter assembly 100. The needle is configured to be deformable from an un-deformed first condition when the support member 120 is in the first position, to a deformed second condition when the support member 120 is in the second position. In the deformed second condition, the needle is in an energised condition. Preferably, the needle is formed from a pseudo-elastic material or a superelastic material, details of which are described herein with reference to FIG. 10 to FIG. 12.

In this way, the intermediate portion is deformable by the base member 110, typically by the first guide element 112, as the support member 120 moves from the first position to the second position.

The support member 120 includes a latch element 130 configured to lock the support member 120 to the base member 110 in the second position. That is, the support member 120 is configured to clip-fit to the base member 110. In this way, a locking engagement is provided by moving the support member 120 from the first position, in which the support member 120 is unlocked from the base member 110, to the second position, in which the support member 120 is locked to the base member 110. A convenient, reliable locking means is thereby provided.

In the example shown, the latch element 130 includes a pair of tangs 132 and a pair of projecting elements 134. Each projecting element 134 of the pair of projecting elements 134 is disposed on either side of the longitudinal axis 150. Each projecting element 134 is mounted to the body portion 122 to extend away from the body portion 122 in a direction parallel to the longitudinal axis 150. As described in more detail herein, the projecting element 134 may be configured to contactingly engage the receiving portion 106 of the base member 110 as the support member 120 moves from the first position to the second position.

Each tang 132 of the pair of tang 132 is disposed on either side of the longitudinal axis 150. Each tang 132 is mounted to the body portion 122 to extend away from the body portion 122 in a direction parallel to the longitudinal axis 150. Each tang 132 is configured to be resiliently deflectable as the support member 120 moves towards the second position. In particular, the free end of each tang 132 is configured to be deflectable towards the longitudinal axis 150.

In the example shown, a first projecting element 134 and a first tang 132 are disposed on a first side of the longitudinal axis 150, and a second projecting element 134 and a second tang 132 are disposed on a second side of the longitudinal axis 150. The tangs are disposed outward of the projecting elements relative to the longitudinal axis 150.

Other suitable arrangements of tangs and/or projecting elements may be provided on the support member so as to cooperatingly engage with the base member 110 of the needle inserter assembly 100, and lock the support member 120 to the base member 110. For examples, a tang may be mounted to a projecting element. Optionally, the latch element may be configured to selectively lock the support member to the base member. In this way, the latch element may be released from the base member by the user, for example by actuating or deforming a tang or other projecting element to disengage the locking engagement between the support member and the base member.

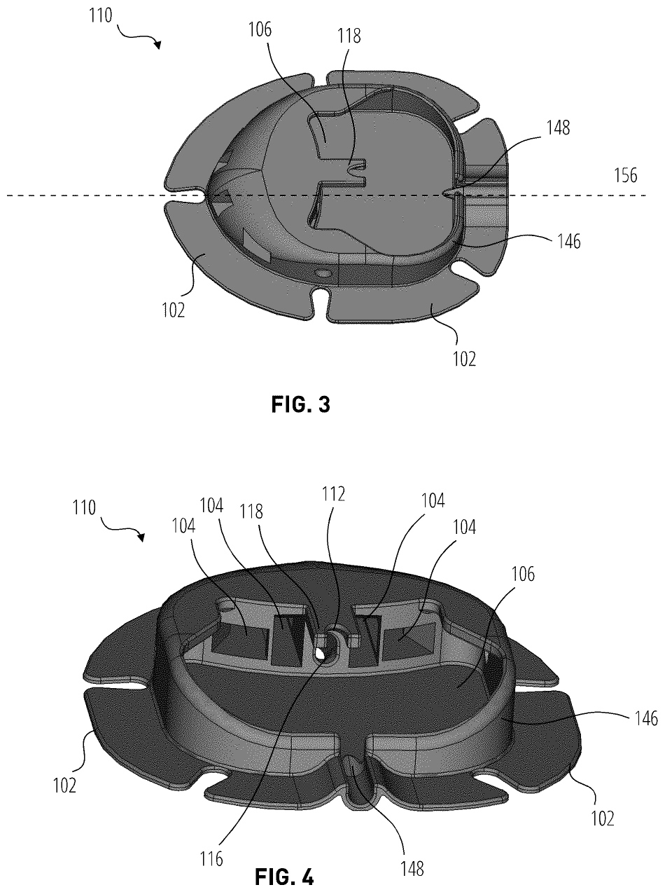

Referring specifically to FIG. 3 and FIG. 4, the base member 110 is shown. The base member 110 includes a receiving portion 106 for receivingly engaging the support member 120. That is, the receiving portion 106 includes a complementary shape to engage the support member 120 when it is fixed to the base member 110 in the second position.

The base member 110 comprises an engaging surface. The engaging surface is formed on the lower surface of the base member 110 and is configured to contact the soft tissue of a patient during insertion of the tip of the needle into the soft tissue. The opening 114 is located in the engaging surface so that the tip of the needle projects from the engaging surface in the second position.

The base member 110 includes a wall portion 146 partially surrounding and defining the receiving portion 106. The wall portion 146 includes a notch 148 to allow tubing 136 coupled to the support member 120 to extend away from the support member 120 without being impeded by the base member 110.

The base member 110 includes plurality of flanges 102 extending outwardly from the base member 110. The plurality of flanges 102 may be moveable with respect to the main housing of the base member 110 so that the base member 110 may be adapted to optimise engagement with the soft tissue of a patient. That is by moving one or more of the plurality of flanges 102, the lower surface of the base member 110 may be adapted to contactingly engage soft tissue that is uneven or is not flat.

The base member 110 includes a bore 116. The bore 116 extends through the base member 110 to the opening 114 provided on the lower surface of the base member 110. In this way, the first guide element 112 includes a surface, that is an inner surface, of the bore 116.

The bore 116 extends to the opening 114 along an insertion axis 152. The insertion axis 152 is oriented in the second direction at an acute angle to the engaging surface. In this way, the bore 116 is arranged to constrain the distal portion 128 of the needle along the insertion axis 152 as the support member 120 is moved relative to the base member 110 from the first position to the second position.

In the example, the first guide element 112 of the base member 110 also includes an arm portion 118. The arm portion 118 is arranged to extend over the receiving portion 106 of the support member 120. The arm portion 118 is aligned with the insertion axis 152 of the bore 116. Thus, in use, the arm portion 118 engages the distal portion 128 of the needle as it moves towards the second position. In this way, the arm portion 118 is configured to receivingly guide the distal portion 128 toward the bore 116 of the first guide element 112.

The receiving portion 106 of the base member 110 includes a pair of recessed cavities 104. Each recessed cavity 104 is positioned and oriented to receive a portion of the support member 120. In the example, each recessed cavity 104 extends into the housing 108 of the base member 110. Each recessed cavity 104 extends in the first direction, that is in the direction of the base member longitudinal axis 156.

A first, inner pair of recessed cavities 104 is disposed on either side of the base member longitudinal axis 156. A second, outer pair of recessed cavities 104 is disposed on either side of the base member longitudinal axis 150.

Each recessed cavity 104 of the inner pair of recessed cavities 104 engages a projecting element 134 as the support member 120 is moved towards the second position. In this way, the receiving portion 106 is configured to contactingly engages the support member 120 to deflect the body portion 122 towards the first direction. The receiving portion 106 is thereby configured to contactingly engage the support member 120 to deflect the body portion 122 at an angle to the second direction.

Each recessed cavity 104 of the outer pair of recessed cavities 104 engages a tang 132 of the support member 120. A detent formation at the distal end of each cavity provides a complementary engagement with the tang to lock the support member 120 to the base member 110.

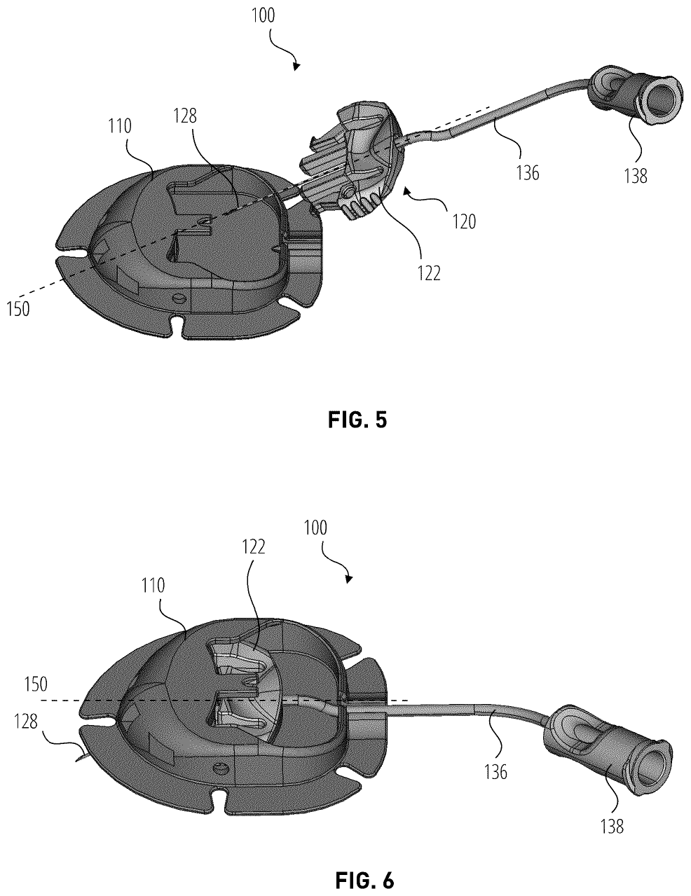

Referring now to FIG. 5, the support member 120 is shown in the first position, oriented to engage with the base member 110. In the first position, the support member 120 is oriented relative to the base member 110 so that its longitudinal axis 150 extends in the second direction.

In the first position, the longitudinal axis 150 is oriented to align along the insertion axis 152 of the first guide element 112. In this way, the longitudinal axis 150 of the support member 120 is oriented at an angle to the engaging surface corresponding to the angle between the insertion axis 152 and the engaging surface.

In the first position, the proximal portion 124, the intermediate portion 126, and the distal portion 128 of the needle are aligned both along the longitudinal axis 150 of the support member 120 and along the insertion axis 152.

In addition, the longitudinal axis 150 of the support member 120 is oriented at a second acute angle to the longitudinal axis 156 of receiving portion 106 of the base member 110.

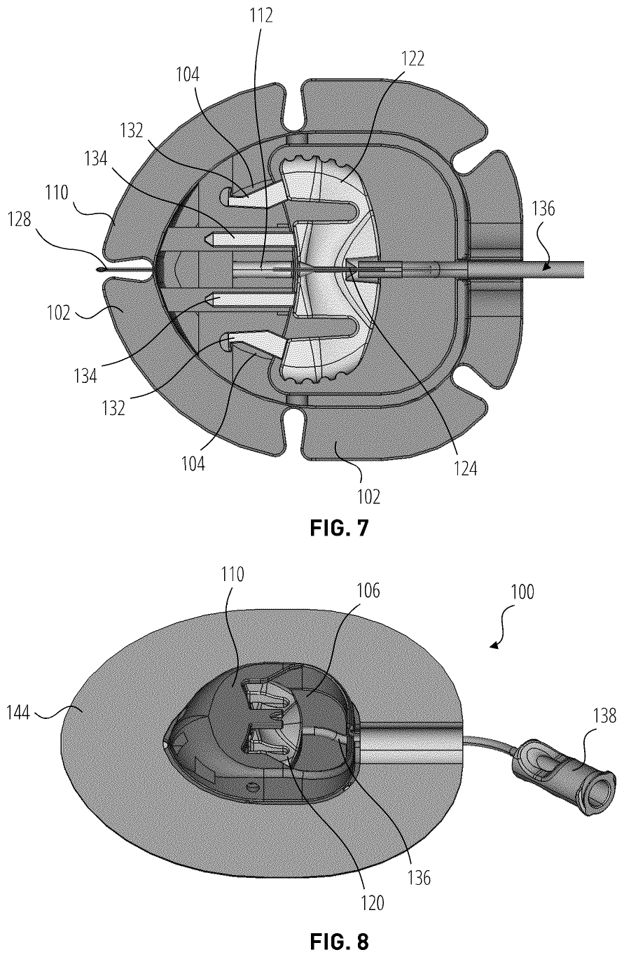

Referring additionally to FIG. 6 and FIG. 7, the support member 120 is shown mounted to the base member 110 in the second position. In the second position, the support member 120 is mounted to the base member 110 with the longitudinal axis 150 extending in a first direction. In the first direction, the longitudinal axis 150 of the support member 120 is oriented parallel to the longitudinal axis 156 of the receiving portion 106 of the base member 110.

In the second position, the support member 120, and thereby the proximal portion 124 of the needle, is at an acute angle to the insertion axis 152.

In the second position, the intermediate portion 126 and the distal portion 128 are aligned along the insertion axis 152. The intermediate portion 126 and the distal portion 128 are aligned at an acute angle to both the longitudinal axis 150 of the support member 120 and the longitudinal axis 156 of the base member 110.

In the second position, each of the tangs 132 and the projecting elements 134 of the support member 120 is received within a recessed cavity 104 of the base member 110. In particular, the tangs 132 are lockingly engaged with a respective detent formation.

Referring additionally to FIG. 8, the needle inserter assembly 100 is illustrated in the second position mounted to a patient (not shown). The support member 120 is lockingly engaged with the base member 110 so that the tubing 136 extends through the notch 148 provided in the wall portion 146 of the base member 110. The protective cover 144 is applied over the flanges 102 of the base member 110 and over the tubing 136, securing the needle inserter assembly 100 to the patient and forming a barrier to ingress of dirt or contamination to the needle incision in the soft tissue.

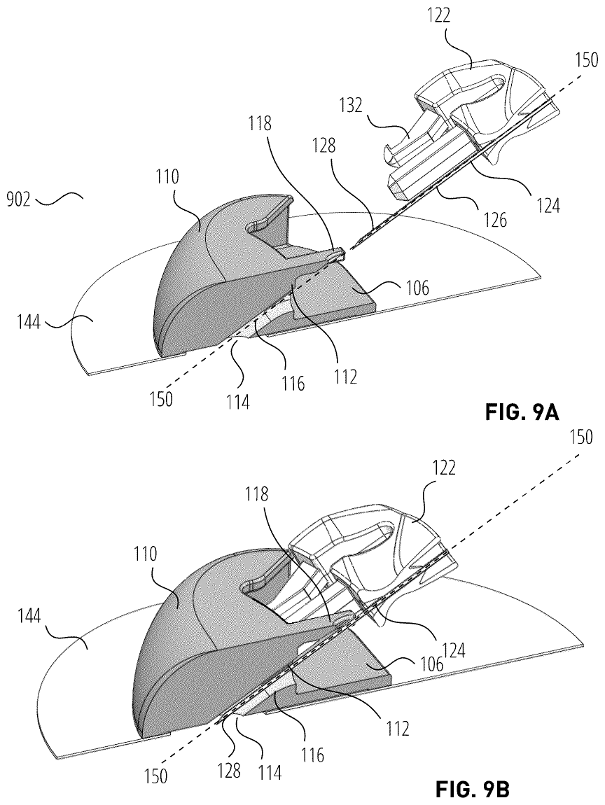

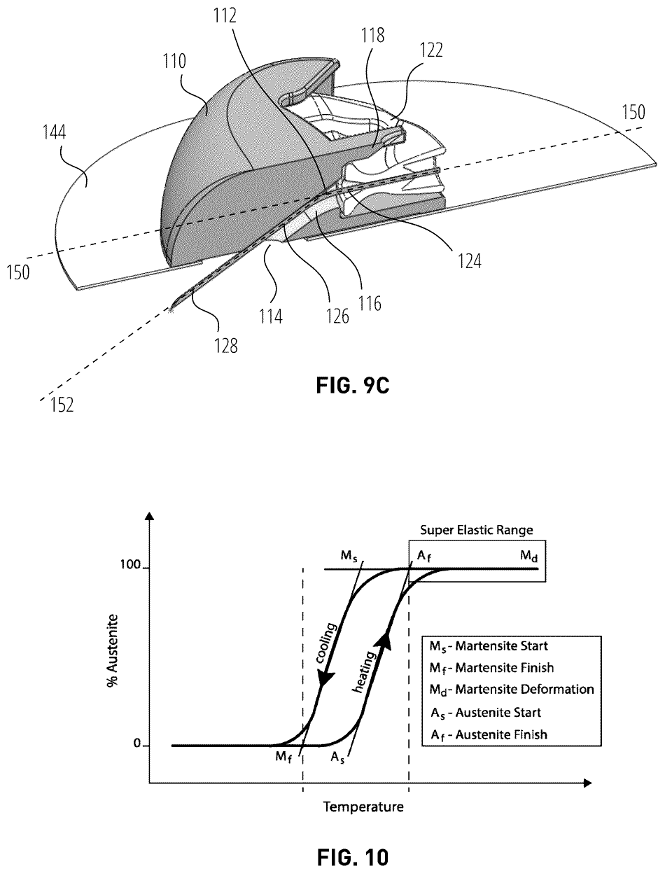

Referring now to FIG. 9A to FIG. 9C, the needle inserter assembly 100 is shown in use.

Referring particularly to FIG. 9A, the needle inserter assembly 100 is shown in the first position with the support member 120 oriented for engagement with the body portion 122 and the tip of the distal portion 128 beginning to engage the first guide element 112. The proximal portion 124, intermediate portion 126, and the distal portion 128 of the needle are aligned in a second direction, that is, aligned along the insertion axis 152. In this way, the needle is oriented to be easily received by the first guide element 112, without laterally deflecting the distal portion 128 of the needle. As will be appreciated, the provision of the first guide element 112 also allows for orientations in which the needle is not aligned ideally with the insertion axis 152 because the first guide element 112, via the arm portion 118 and/or the bore 116 are arranged to engage the distal portion 128 and guide it along the insertion axis 152 as the support member 120 is urged into engagement with the base member 110.

Referring now to the FIG. 9B, the support member 120 is shown in a position intermediary between the first position and the second position. The distal portion 128 and the intermediate portion 126 of the needle are substantially engaged with the first guide element 112. At this point, the projecting elements 134 have made contact with the receiving portion 106 of the base member 110 so that the support member 120 can be urged no further in the second direction and, instead subsequent urging deflects the support member 120 towards the first direction. Stated differently, further engagement of the support member 120 with the base member 110 deflects the longitudinal axis 150 of the support member 120 towards the orientation of the base member longitudinal axis 156.

The first guide element 112 is configured so that, as the support member 120 moves from the first position towards the second position, the first guide element 112 contactingly engages the needle to guide the tip towards the opening 114 in a second direction, wherein the second direction is at an angle to the first direction. Stated differently, as the support member 120 moves from the first position to the second position, the first guide element 112 guides the tip at an angle to the longitudinal axis of the support member 120.

The first guide element 112 guides the tip along the bore 116 and towards the opening 114 on the engaging surface of the base member 110. The tip is guided towards the soft tissue along the insertion axis 152. The tip is guided at a fixed angle relative to the soft tissue.

Referring now to FIG. 9C, the support member 120 is shown in the second position. The distal portion 128 projects from the opening 114. The needle projects into the soft tissue along the direction of the insertion axis 152. In this way, the needle is urged into the soft tissue without lateral deflection.

The needle is urged along the insertion axis 152 regardless of the orientation of the support member 120 relative to the base member 110. In this way, the tip of the needle is inserted into the soft tissue along a predetermined insertion axis, independent from the means of locking engagement. The tip is secured with the needle inserter assembly in the second position so that the needle inserter assembly is convenient to use in a space-efficient manner.

The needle inserter assembly described with reference to FIG. 1 to FIG. 9C thereby provides a convenient method of inserting a needle having a lumen for distributing fluid into, or removing fluid from a soft tissue of a patient.

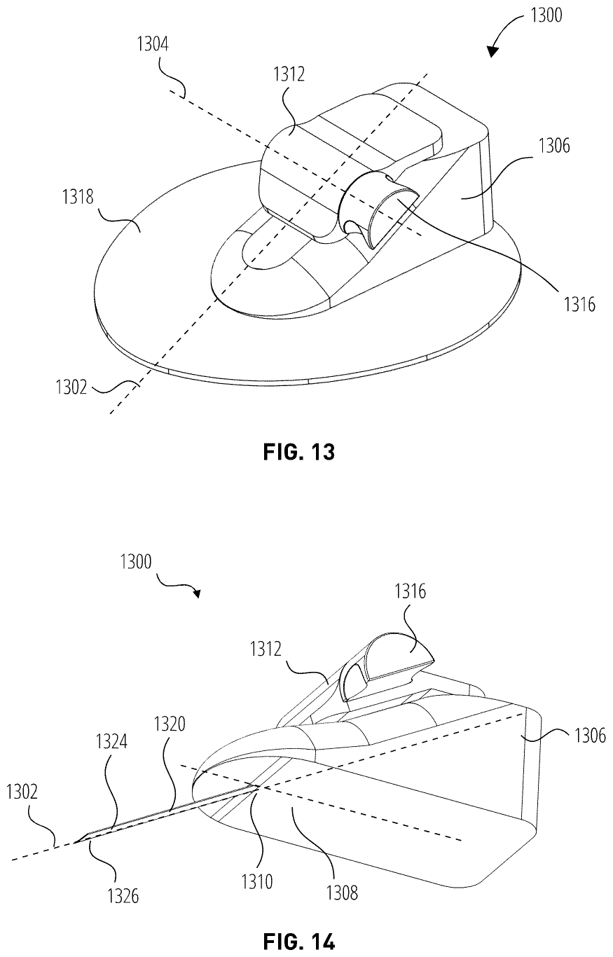

Referring now to FIG. 13 to FIG. 16, there is shown an example needle inserter assembly 1300. The needle inserter assembly 1300 includes a base member 1306 and a needle 1320. The needle 1320 includes a lumen, a proximal portion 1322, and a distal portion 1324 having a tip 1326 configured to facilitate insertion into a soft tissue of a patient.

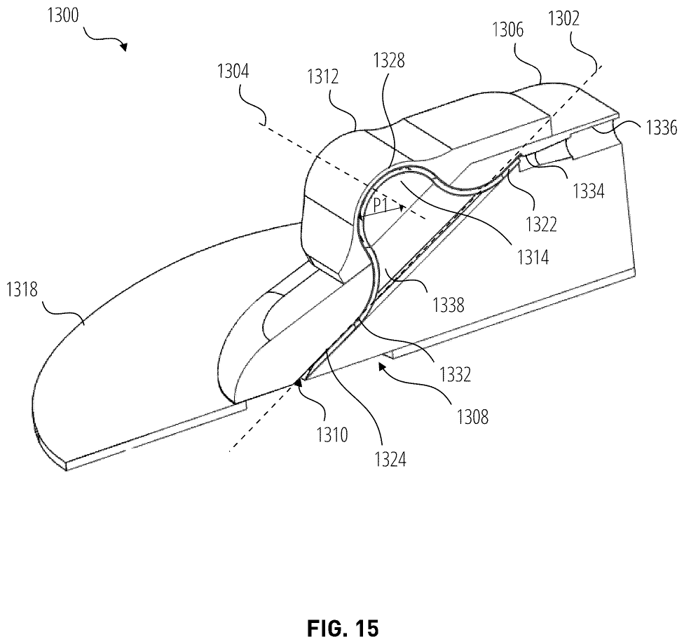

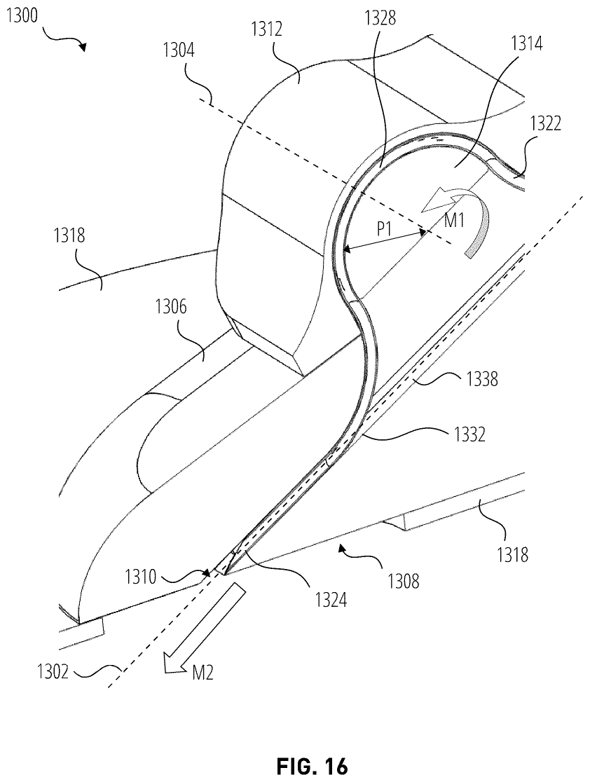

The needle 1320 is operably mounted within the needle inserter assembly 1300 so that the distal portion 1324 is moveable from a first position, in which the distal portion 1324 extends through an opening 1310 provided through the base member 1306, to a second position in which the distal portion 1324 is retracted within the base member 1306, as is described herein. The first position is shown and described particularly with reference to FIG. 14. The second position is shown and described particularly with reference to FIG. 15 and FIG. 16.

The needle inserter assembly 1300 includes a first engaging element 1314 mounted thereto. In the example, the first engaging element 1314 is rotatably engaged with a cover 1312 that is releasably mounted to the base member 1306. In this way, the first engaging element 1314 is rotatably mounted within the needle inserter assembly 1300 and configured to rotate around an element axis 1304.

The first engaging element 1314 includes an actuator 1316. The actuator 1316 is arranged external to the cover 1312 when the first engaging element 1314 is rotatably engaged with the cover 1312. In this way, the actuator 1316 is operably engageable by a user to manually rotate the first engaging element around the element axis. Optionally, an actuator may be operably engaged with any suitable means to drive the first engaging element, such as an electromechanical motor with a switch.

As shown in more detail with reference to FIG. 15, the element axis 1304 is disposed at an angle to a needle axis. In the example, the element axis 1304 is disposed at an angle perpendicular to the needle axis.

The needle inserter assembly 1300 includes an adhesive patch 1318. The adhesive patch 1318 is suitably tacky to affix a lower surface of the base member 1306 to the soft tissue of the patient. With the needle inserter assembly 1300 affixed to the patient, the needle 1320 may be mounted to the needle inserter assembly 1300 that is operated as described herein, so that the needle 1320 is inserted into the soft tissue of the patient. As will appreciated, the needle inserter assembly 1300 may be affixed or otherwise secured to the patient without an adhesive patch, instead using other suitable means.

The adhesive patch 1318 includes an aperture extending therethrough. The adhesive patch 1318 is suitably positioned to align with the opening 1310 on the lower surface of the base member 1306 when the adhesive patch 1318 is affixed to the lower surface.

The needle inserter assembly 1300 may optionally include a protective cover (not shown) mountable to, or around, a perimeter surface of the base member 1306. The protective cover may be mounted to the base member 1306 after the base member 1306 is affixed to a patient's soft tissue, enclosing a perimeter surface of the base member 1306 against the soft tissue. The protective cover thereby reduces ingress of dirt or contamination between the soft tissue and the needle inserter assembly 1300 during use.

Referring specifically to FIG. 14, there is shown the example needle inserter assembly 1300 of FIG. 13 with the needle 1320 disposed with the distal portion 1324 in the first position. The needle inserter assembly 1300 is shown without the adhesive patch 1318.

The base member 1306 of the needle inserter assembly 1300 includes an engaging surface 1308 disposed to contact the soft tissue of a patient during insertion of the tip of the needle into the soft tissue. The engaging surface 1308 includes an opening 1310 located therein.

In the first position, the distal portion 1324 of the needle 1320 extends through the opening 1310 of the base member 1306. The distal portion 1324, including the tip 1326, extends through the opening 1310 along an insertion axis 1302. In the example shown, the distal portion 1324 extends out of the opening 1310 along the insertion axis 1302, wherein the insertion axis 1302 is disposed relative to the engaging surface 1308 at an angle of 30°.

As will be understood, the insertion axis 1302 may be provided at any suitable angle relative to the engaging surface 1308, for example the insertion axis may be disposed at an angle in the range of from 10° to 80°, preferably 20° to 60°, more preferably 30° to 45°.

Referring specifically to FIG. 15, there is shown a cross-sectional view of the example needle inserter assembly 1300 of FIG. 13 with the needle 1320 disposed with the distal portion 1324 in the second position. The needle inserter assembly 1300 is shown with the adhesive patch 1318.

The base member 1306 of the needle inserter assembly 1300 includes a bore 1332 extending from an upper surface 1338 of the base member 1306 to the opening 1310. The bore 1332 extends from the upper surface 1338 to the opening 1310 along the direction of the insertion axis 1302. In this way, the bore is disposed at an angle of 30° relative to the engaging surface.

The needle 1320 is arranged so that, in moving between the first position and the second position, the distal portion 1324 is reciprocally moveable within the bore 1332. The diameter of the bore is only marginally greater than the diameter of the needle 1320. In this way, the bore 1332 constrains the distal portion 1324 to move along the insertion axis 1302 when moving between the first position and the second position. Optionally, the bore may be configured to constrain the distal portion 1324 of the needle 1320 in any suitable manner, for example using a series of guide elements arranged along a bore or cavity in order to direct guide the distal portion 1324 along the insertion axis 1302.

The needle inserter assembly 1300 further includes a port 1334 configured to receive the proximal portion 1322 of the needle 1320 and fluidly connect the needle 1320 to a fluid channel 1336 to distribute fluid into, or remove fluid from, the soft tissue of a patient. The proximal portion 1322 may be fixedly mounted to within the needle inserter assembly 1300, for example, fixedly mounted to the port 1334. In this way, the proximal portion 1322 is arranged to remain static as the needle 1320 moves the distal portion 1324 between the first position to the second position. In particular, the fixed proximal portion 1322 ensures the elastically deformable needle 1320 pushes away from the proximal portion 1322 to provide a maximal urging force to move the distal portion 1324 back to the first position.

The needle inserter assembly 1300 may include a tubing (not shown) mounted to fluid channel 1336 in the base member 1306, and fluidly connected to the lumen of the needle 1320. The proximal portion 1322 is fluidly connected to the lumen via the port 1334. The tubing and fluid channel 1336 each include a respective lumen to for delivery or removal of fluid to the needle 1320 when mounted to the port 1334.

The needle 1320 of the needle inserter assembly 1300 includes an intermediate portion 1328 disposed between the proximal portion 1322 and the distal portion 1324.

The needle inserter assembly 1300 includes a first engaging element 1314. The first engaging element 1314 is configured to contactingly engage the needle 1320 and displace the intermediate portion 1328 of the needle 1320 to move the distal portion 1324 of the needle 1320 from the first position to the second position.

In the example, the first engaging element 1314 is a semi-circular rod. That is, a rod with a semi-circular profile extending along a rod axis. The first engaging element 1314 is arranged with the rod axis oriented along the element axis 1304, perpendicular to the insertion axis 1302. The first engaging element 1314 is thereby configured to rotate around the element axis 1304 between a first rotational position and second rotational position corresponding to opposing rotational positions of the first engaging element 1314 relative to the needle 1320.

In the first rotational position the flat surface of the first engaging element 1314 is oriented towards the needle 1320. The needle 1320 thereby lies against the flat surface of the first engaging element 1314 so that the needle is substantially straight. That is, the distal portion 1324, the proximal portion 1322 and an intermediate portion 1328 all lie along a needle axis. The needle axis is aligned with the insertion axis 1302. The distal portion 1324 of the needle 1320 is in the first position.

In the second rotational position the first engaging element 1314 is disposed in an opposing orientation. In the second rotational position the curved surface of the first engaging element 1314 is oriented towards the needle 1320. The intermediate portion 1328 of the needle 1320 is displaced away from the needle axis to follow an arcuate path around the first engaging element 1314. The proximal portion 1322 and the distal portion 1324 lie along the needle axis, that is aligned along the insertion axis 1302. Due the displacement of the intermediate portion 1328, the distal portion 1324 of the needle 1320 is retracted into the base member 1306 towards the proximal portion 1322. The distal portion 1324 of the needle 1320 is in the second position.

With the distal portion 1324 in the second position, the intermediate portion is deflected around an arcuate path by the first engaging element 1314. In the example, the arcuate path has a pitch radius P1 of 3 mm.

Referring additionally to FIG. 16, the example needle inserter assembly 1300 is shown in use, so that the needle inserter assembly is affixed to a patient's soft tissue using the adhesive patch 1318.

The first engaging element 1314 has been rotated to the second rotational position so that the curved surface has engaged the elastically deformable needle 1320 and displaced the intermediate portion 1328 to move the distal portion 1324 to its second position. The distal portion 1324, including the tip 1326, is retracted through the opening 1310 into the bore 1332 of the base member 1306.

The opening 1310 is proximal to the soft tissue. The needle inserter assembly 1300 is primed for use to insert the tip 1326 into soft tissue. Stated differently, displacing the intermediate portion 1328, energises the elastically deformable needle 1320. The needle 1320 is disposed in an energised state by the first engaging element 1314.

To use the needle inserter assembly 1300, the first engaging element 1314 is rotated to the first rotational position, as indicated by arrow M1. Rotation of the first engaging element 1314 to the first rotational position releases the needle to recover. In releasing the needle 1320 from the energised state, the elastic resilience of the needle 1320 moves the intermediate portion 1328 towards the needle axis, thereby enabling the needle 1320 to recover an un-deformed state. In this way, the needle 1320 provides an urging force to move the distal portion 1324 back towards the first position, indicated by arrow M2.

Recovery of the needle 1320 from the energised state to the un-deformed state urges the distal portion 1324 along the insertion axis 1302, to move the distal portion 1324 along the bore 1332 and out of the opening 1310. The urging force thereby urges the tip 1326 against the soft tissue, forming an incision in the soft tissue.

In the example shown, in the first position, the distal portion 1324 is retracted into the base member 1306 only a small distance beyond the opening 1310. When the first engaging element 1314 releases the intermediate portion 1328 of the needle 1320, the urging force provided to the distal portion is a maximal urging force when the distal portion is in the second position. In this way, the maximal urging force is provided as the tip 1326 engages and pierces the soft tissue, providing an easier insertion of the needle 1320 into the soft tissue.

Furthermore, as the distal portion 1324 is constrained by the bore 1332 to move only along the insertion axis 1302, the distal portion 1324 does not move laterally to the desired insertion axis 1302. The urging force is thereby directed more effectively to urge the tip 1326 to pierce and then pass into the soft tissue.

Although not shown in the example, the needle inserter assembly 1300 may optionally include a second engaging element configured to contactingly engage the needle to selectively displace the intermediate portion towards the needle axis. In this way, the second engaging element may provide a secondary urging force to move the distal portion at least partially back towards the first position.

The needle inserter assembly 1300 described with reference to FIG. 13 to FIG. 16 thereby provides a convenient method of inserting a needle having a lumen for distributing fluid into, or removing fluid from a soft tissue of a patient. According to a first step, the method includes providing a needle, wherein the needle comprises a proximal portion and a distal portion having a tip configured to facilitate insertion into a soft tissue of a patient.

According to a second step, the method includes providing a base member with needle operably mounted therein.

According to a third step, the method includes elastically deforming the needle to move the distal portion to a second position, in which the distal portion is retracted within the base member.

According to a fourth step, the method includes engaging the base member on the soft tissue, and releasing the distal portion from the second position so that the needle provides an urging force to move the distal portion back towards a first position in which the distal portion extends from the base member.

In certain embodiments, the needle may be superelastic, i.e. the needle may be formed from a superelastic material, for example a superelastic alloy. Superelasticity, also referred to as pseudo-elasticity, is an elastic response exhibited by certain materials to an applied stress. Superelasticity occurs when an applied stress induces an austenite to martensite phase transformation in the material and a corresponding strain, which is recoverable by removing the applied stress. Certain superelastic materials exhibit recoverable strains of up to 11%, which is significantly greater than more conventional materials. For example, 316 stainless steel (16% chromium, 10% nickel and 2% molybdenum), which is commonly used in medical applications, exhibits recoverable strains of approximately 0.5%. A superelastic alloy used in medical applications is nickel titanium alloy, commonly referred to as Nitinol. The needle may be formed of Nitinol. As superelasticity is a stress-induced phase transformation from austenite to martensite, for the needle to exhibit optimum superelasticity it may be formed of so-called austenitic (or superelastic) Nitinol, in which Nitinol will be substantially fully austenitic, i.e. the primary crystalline structure of the alloy is austenite. Nitinol will remain substantially fully austenitic above its martensite start temperature Mₛ. This is important to note because an austenite to martensite phase transformation can be induced by cooling as well by applied stress.

Referring now to FIG. 10, there is shown a temperature-induced phase transformation of a Nitinol alloy between the austenitic and martensitic phases, in which austenite is stable at relatively higher temperatures and martensite is stable at relatively lower temperatures. Heating Nitinol beyond its austenite start temperature Aₛ causes it to transform to the austenitic phase. Nitinol will be substantially fully austenitic once heated above its austenite finish temperature A_{f}. As above, it is in this substantially fully austenitic phase that Nitinol will exhibit optimum superelasticity, allowing the needle when formed of Nitinol to elastically deform, i.e. flex/bend, through a relatively broad range of stresses without causing permanent deformation. The optimal superelastic range (also referred to as a superelastic window) between the austenite finish temperature A_{f} and the martensite deformation temperature M_{d}, is highlighted in FIG. 10. From the substantially fully austenitic phase, cooling Nitinol beyond its martensite start temperature Mₛ causes Nitinol to transition to the martensitic phase. Below its martensite finish temperature M_{f} Nitinol will be substantially fully martensitic.

The graph in FIG. 10 also shows that Nitinol exhibits thermal hysteresis, i.e. the temperature at which martensite transforms to austenite is not that at which austenite transforms to martensite. The hysteresis may be approximately 20-30° C. (i.e. A_{f}−M_{f}) for fully annealed Nitinol alloys, such as those used in medical device applications. It is known that a greater thermal hysteresis will yield a greater mechanical hysteresis. The significance of its thermal hysteresis is that Nitinol remains in its austenitic phase when cooled beyond its austenite finish temperature A_{f}. This means a needle when formed of austenitic Nitinol will remain superelastic above its martensite start temperature Mₛ, which may therefore be the critical transformation temperature when selecting an alloy forming the needle.

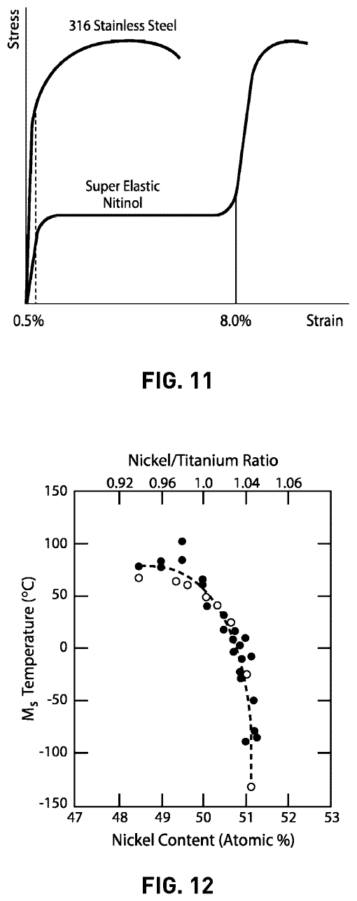

Referring now to FIG. 11, there is shown a graph comparing the behavior of a needle formed of 316 stainless steel with a needle formed of nitinol. As is evident from the figure, the nitinol needle has a large range of elastic deformation in which increased strain does not significantly increase the stresses within the needle. In the illustrated form, stresses begin to rise at about 8% strain. As such, it may be advisable to maintain the strain associated with the needle below about 6% in order to ensure that the needle remains within its elastic deformation range.

Referring now to FIG. 12, there is shown a correlation between the martensite start temperature Mₛ and the composition of Nitinol. Since the martensite start temperature Mₛ is correlated with the nickel/titanium ratio it can be predetermined, for example a Nitinol alloy can be selected having a martensite start temperature Mₛ from approximately −130° C. to approximately +110° C.

As above, certain embodiments of the invention may have particular application for use in devices for CSII therapy, in which the devices may be worn by a patient such that at least the tip and the distal portion of the needle is placed in the patient's soft tissue. Due to the superelasticity of Nitinol, the needle may reduce tissue damage by exhibiting a relatively high degree of flexibility. However, if the martensite percentage of the material forming the tip begins to rise, exposure to stress may cause it undergo plastic deformation, resulting in an irregular, uncontrolled shape change. This may lead to tissue damage. While the patient's body heat may aid in maintaining Nitinol in its austenitic phase, certain embodiments of the needle may be placed in tissue to a relatively low depth, such as approximately 3 to 4 mm. As such, cold temperatures experienced by the patient may cause a temperature-induced phase transformation of the Nitinol alloy forming the needle to its martensitic phase, i.e. if the martensite start temperature Mₛ is too high. Thus, in certain embodiments, the needle may be formed of a Nitinol alloy selected to remain substantially fully austenitic (for example, at least 95% austenitic) in temperatures likely to be experienced by the patient to reduce the risk of the needle becoming martensitic.

As indicted in FIG. 10, superelasticity is exhibited up to the martensite deformation temperature M_{d}, which corresponds to the greatest temperature at which it is possible to stress-induce the formation of martensite. Above the martensite deformation temperature Mu the response to stress is non-elastic deformation of the austenitic microstructure, since martensite can no longer be formed, and thus permanent deformation. In other words, above the martensite deformation temperature M_{d} the Nitinol will deform plastically and irreversibly.

The effective superelastic window of the Nitinol may be increased to span more than 200° C., with a significant reduction in temperature-stress sensitivity, by subjecting the material to a controlled process. The Nitinol may, for example, be subjected to an annealing process or treatment, e.g. an isobaric annealing process or treatment in Argon at approximately 350° C. to 400° C. In this way, the martensite deformation temperature M_{d} of the material is increased and the effective superelastic window or range may be increased or widened.

In view of the above, the needle may be formed of a Nitinol alloy having been subjected to an isobaric annealing treatment in order to increase the effective superelastic window in order to ensure that elastic deformation through a relatively broad range of stresses is possible. In this way the needle can be manufactured from Nitinol that elastically bends without permanent deformation of the needle.

The needle may also be formed of a Nitinol alloy having an austenite finish temperature A_{f} in the range of approximately −15° C. to approximately 20° C. in order to ensure that the needle has good superelastic properties at body temperature (approximately 37° C.).

More generally, the needle may be formed of a Nitinol alloy selected such that the needle, or part therefore, exhibits selected properties and/or characteristics with reference to a particular temperature to be experienced by the needle. In certain embodiments, the particular temperature may be body temperature (approximately 37° C.) or room temperature (for example, approximately 20° C. to 22° C.).

FIG. 12 also shows that Nitinol is typically composed of approximately 50 to 51% nickel by atomic percent, though as shown in FIG. 4 compositions outside this range are known, and various compositions may be suitable for forming the needle. In certain embodiments, an atomic ratio of nickel to titanium is between 1.01 and 1.05. In certain embodiments, an atomic ratio of nickel to titanium is between 1.02 and 1.04.

The needle formed of austenitic (or superelastic) Nitinol may exhibit greater flexibility than a similarly-configured needle formed of a more conventional material, such as stainless steel. The skilled reader will understand that the range of superelasticity for a particular composition of Nitinol depends largely upon its nickel/titanium ratio. In certain embodiments, the needle may be formed of Nitinol having a nickel/titanium ratio selected to provide the needle with particular properties and/or characteristics. The skilled reader will also understand that the range of superelasticity of the Nitinol also depends on how the material has been processed. In certain embodiments, the needle may be formed of Nitinol that has been processed or treated in a controlled way in order to provide the needle with particular properties and/or characteristics.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A needle inserter assembly comprising:
a support member comprising a body portion and a needle, wherein the needle comprises a lumen, a distal portion having a tip configured to facilitate insertion into a soft tissue of a patient, and a proximal portion mounted to the body portion so that the proximal portion extends along a longitudinal axis of the body portion; and
a base member comprising a receiving portion configured to receivingly engaging the support member as the support member is moveable from a first position, in which the tip engages a first guide element within the base member, to a second position in which the tip extends out of an opening provided through the base member;
wherein, in the second position, the support member is mounted to the base member with the longitudinal axis oriented in a first direction; and
wherein the first guide element is configured so that, as the support member moves from the first position towards the second position, the first guide element contactingly engages the needle to guide the tip towards the opening in a second direction, wherein the second direction is at an angle to the first direction.

2. A needle inserter assembly according to claim 1, wherein the receiving portion is configured to receivingly engage the support member with the longitudinal axis oriented in a second direction as the support member is moved from the first position towards the second position.

3. A needle inserter assembly according to claim 1 or claim 2, wherein as the support member moves from the first position to the second position, the first guide element contactingly engages an intermediate portion of the needle.

4. A needle inserter assembly according to any one of claims 1 to 3, wherein the support member is substantially planar along the longitudinal axis.

5. A needle inserter assembly according to any one of claims 1 to 4, wherein the support member comprises a port and the proximal portion is fluidly connected to the port.

6. A needle inserter assembly according to any one of claims 1 to 5, wherein as the support member moves from the first position to the second position, the first guide element guides the tip at an angle to the longitudinal axis of the support member.

7. A needle inserter assembly according to any one of claims 1 to 6, wherein the support member comprises a latch element configured to releasably lock the support member to the base member in the second position.

8. A needle inserter assembly according to claim 7, wherein the latch element includes at least one tang extending from the body portion in the lengthwise direction, wherein each tang is configured to be resiliently deflectable as the support member moves towards the second position, and, optionally, wherein a tang is mounted to a projecting element.

9. A needle inserter assembly according to any one of claims 1 to 8, wherein the support member includes at least one projecting element mounted to the body portion to extend away from the body portion and wherein the projecting element is configured to contactingly engage the receiving portion of the base member as the support member moves from the first position to the second position.

10. A needle inserter assembly according to any one of claims 1 to 9, wherein the base member includes a bore extending through the base member to the opening, and wherein first guide element comprises an inner surface of the bore.

11. A needle inserter assembly according to any one of claims 1 to 10, wherein the bore extends to the opening along an insertion axis and wherein the insertion axis is oriented in the second direction.

12. A needle inserter assembly according to any one of claims 1 to 11, wherein the base member comprises an engaging surface configured to contact the soft tissue of a patient during insertion of the tip of the needle into the soft tissue, and wherein the opening is located in the engaging surface.

13. A needle inserter assembly according to claim 11, wherein, in the second position the tip extends out of the opening in the second direction, and wherein the second direction is disposed at an angle relative to the engaging surface in a range of from 20° to 40°, preferably in a range 25° to 35°.

14. A needle inserter assembly according to any one of claims 1 to 13, wherein in the second position, the distal portion is disposed in the second direction and the proximal portion is disposed in the first direction.

15. A needle inserter assembly according to any one of claims 1 to 14, wherein, in the first position, the distal portion and the proximal portion are aligned along the longitudinal axis of the support member.

16. A needle inserter assembly according to any one of claims 1 to 15, wherein the needle includes an intermediate portion and, in the second position, the intermediate portion is deformed by the first guide element and, optionally, wherein, in the first position, the distal portion, the intermediate portion, and the proximal portion are aligned along the longitudinal axis of the support member.

17. A needle inserter assembly according to any one of claims 1 to 16, wherein the receiving portion comprises at least one recessed cavity for receiving at least a portion of the support member and, optionally, wherein each recessed cavity extends inwardly from the receiving portion in the first direction.

18. A needle inserter assembly according to any one of claims 1 to 17, wherein the needle is elastically deformable.

19. A needle inserter assembly according to any one of claims 1 to 18, wherein the needle is formed from a pseudo-elastic material or a superelastic material.

20. A needle inserter assembly according to claim 19, wherein the needle is formed from a nitinol material, typically a nitinol material with an austenite finish temperature in the range of from −15° C. to 30° C., preferably in the range of from 0° C. to 20° C.

21. A needle inserter assembly according to any preceding claim, wherein the needle has one or more of:
a wall thickness defined between an outer surface and a lumen surface, wherein the wall thickness is at least 20 μm;
a wall thickness defined between an outer surface and a lumen surface, wherein the wall thickness is less than 400 μm;
an outer diameter, wherein the outer diameter is less than 0.7 mm;
an outer diameter, wherein the outer diameter is at least 0.4 mm;
an inner diameter, wherein the inner diameter is at least 0.2 mm; or
an inner diameter, wherein the inner diameter is less than 0.5 mm.

22. A needle inserter assembly comprising:
a support member including a body portion and a needle, wherein the needle comprises a lumen, a proximal portion mounted to the body portion, and a distal portion having a tip configured to facilitate insertion into a soft tissue of a patient; and
a base member comprising a receiving portion configured to receivingly engaging the support member as the support member is moveable from a first position, in which the tip is retracted within the base member to a second position in which the tip extends out of an opening provided through the base;
wherein the base member includes a first guide element; and
wherein the receiving portion is configured so that, as the support member moves from the first position towards the second position, the first guide element contactingly engages the needle to guide the tip in a first direction towards the opening, and the receiving portion contactingly engages the support member to deflect the body portion at an angle to the first direction.

23. A method of inserting a needle having a lumen for distributing fluid into, or removing fluid from a soft tissue of a patient, the method comprising:
providing a support member of a needle inserter assembly, wherein the support member comprises a body portion and a needle, and wherein the needle comprises a proximal portion mounted to the body portion, and a distal portion having a tip configured to facilitate insertion into a soft tissue of a patient;
engaging a base member of the needle inserter assembly with the soft tissue, wherein the base member comprises a receiving portion configured to receivingly engaging the support member, an opening, and a first guide element;
engaging the support member with the receiving portion of the base member in a first position so that the needle engages the first guide element with the distal portion oriented in a second direction and with the tip of the needle retracted within the base member;
moving the support member in a first direction to a second position, wherein the first direction is at an angle to the second direction, and
guiding the tip of the distal portion in the second direction with the first guide element as the support member is moved towards the second position.

24. The method of claim 23, wherein the step of guiding the tip of the distal portion moves the tip towards and through the opening in the base member.

25. The method of claim 23 or 24, wherein the step of moving the support member from the first position to the second position elastically deforms the needle.

26. The method of any one of claims 23 to 25, the first guide element engages an intermediate portion of the needle.

27. A needle inserter assembly comprising:
a base member; and
a needle, the needle comprising a lumen, a proximal portion, and a distal portion having a tip configured to facilitate insertion into a soft tissue of a patient;
wherein the needle is operably mountable within the needle inserter assembly so that the distal portion is moveable from a first position, in which the distal portion projects through an opening provided through the base member, to a second position in which the distal portion is retracted within the base member; and
wherein the needle is elastically deformable to provide an urging force to move the distal portion back towards the first position when the distal portion is moved towards the second position.

28. The needle inserter assembly according to claim 27, wherein the needle further comprises an intermediate portion disposed between the proximal portion and the distal portion.

29. The needle inserter assembly according to claim 27 or 28, wherein the proximal portion is fixedly mountable within the needle inserter assembly.

30. The needle inserter assembly according to any one of claims 27 to 29, wherein, in the first position, the proximal portion and the distal portion are aligned along a needle axis.

31. The needle inserter assembly according to any one of claims 27 to 30, wherein, in the second position, an intermediate portion of the needle is displaced away from the needle axis.

32. The needle inserter assembly according to any one of claims 28 to 31, further comprising a first engaging element configured to contactingly engage the needle and displace the intermediate portion of the needle to thereby move the distal portion from the first position to the second position.

33. The needle inserter assembly according to claim 32, wherein the first engaging element is rotatably mounted within the needle inserter assembly and configured to rotate around an element axis disposed at an angle to a needle axis, preferably disposed perpendicular to the needle axis, and optionally, wherein the first engaging element comprises an actuator configured to be operably engaged by a user to manually rotate the first engaging element around the element axis.

34. The needle inserter assembly according to any one of claims 28 to 33, further comprising a second engaging element configured to contactingly engage the needle to selectively displace the intermediate portion towards the needle axis and provide a secondary urging force to move the distal portion at least partially back towards the first position.

35. The needle inserter assembly according to any one of claims 27 to 34, wherein the urging force is provided in a direction along the needle at the distal portion.

36. The needle inserter assembly according to any one of claims 27 to 35, wherein the base member comprises an engaging surface configured to contact the soft tissue of a patient during insertion of the tip of the needle into the soft tissue, and wherein the opening is located in the engaging surface.

37. The needle inserter assembly according to any one of claims 27 to 36, wherein, in the first position the at least the tip of the distal portion extends out of the opening along an insertion axis, and wherein the insertion axis is disposed relative to the engaging surface at an angle in the range of from 35° to 80°, preferably 20° to 60°, more preferably 30° to 45°.

38. The needle inserter assembly according to any one of claims 27 to 37, wherein the base member includes a bore extending from an upper surface of the base member to the opening, and wherein the distal portion is reciprocally moveable within the bore, and, optionally, wherein the bore extends from the upper surface to the opening along an insertion axis, and wherein the insertion axis is disposed relative to the engaging surface at an angle in a range of from 35° to 80°, preferably from 20° to 60°, more preferably from 30° to 45°.

39. The needle inserter assembly according to any one of claims 27 to 38, wherein the bore is configured to constrain the distal portion to move along an insertion axis when moving between the first position and the second position.

40. The needle inserter assembly according to any one of claims 27 to 39, further comprising a port configured to receive the proximal portion of the needle and fluidly connect the needle to a fluid channel to distribute fluid into, or remove fluid from, the soft tissue of a patient.

41. The needle inserter assembly according to any one of claims 27 to 40, wherein the urging force provided to the distal portion is a maximal urging force when the distal portion is in the second position.

42. The needle inserter assembly according to any one of claims 27 to 41, wherein the urging force provided to the distal portion when the distal portion is in the second position is in a range of from 1N to 5N, preferably from 2N to 4N, more preferably from 27.5N to 3N.

43. A needle according to any one of claims 27 to 42, wherein the needle is elastically deformable in a range of from greater than 0% to 34%, preferably a range of from greater than 0% to 31%.

44. The needle inserter assembly according to any one of claims 27 to 44, wherein the needle is formed from a pseudo-elastic material or a superelastic material.

45. The needle inserter assembly according to any one of claims 27 to 44, wherein the needle is formed from a nitinol material, typically a nitinol material with an austenite finish temperature in the range of from −40° C. to 30° C., preferably in the range of from 0° C. to 20° C.

46. A needle inserter according to any of claims 27 to 45, wherein the needle has one or more of:
a wall thickness defined between an outer surface and a lumen surface, and wherein the wall thickness is at least 20 μm;
a wall thickness defined between an outer surface and a lumen surface, and wherein the wall thickness is less than 400 μm;
an outer diameter, and wherein the outer diameter is less than 0.32 mm;
an outer diameter, and wherein the outer diameter is at least 0.29 mm;
an inner diameter, and wherein the inner diameter is at least 0.27 mm;
an inner diameter, and wherein the inner diameter is less than 0.30 mm.

47. A method of inserting a needle having a lumen for distributing fluid into, or removing fluid from a soft tissue of a patient, the method comprising:
providing a needle, wherein the needle comprises a proximal portion and a distal portion having a tip configured to facilitate insertion into a soft tissue of a patient;
providing a base member with needle operably mounted therein;
elastically deforming the needle to moving the distal portion to a second position, in which the distal portion is retracted within the base member;
engaging the base member on the soft tissue; and
releasing the distal portion from the second position so that the needle provides an urging force to move the distal portion back towards a first position in which the distal portion extends from the base member.

48. The method of claim 47, wherein the step of elastically deforming the needle provides an elastic deformation of an intermediate portion of the needle in a range of from greater than 0% to 34%, preferably a range of from greater than 0% to 31%.

49. The method of claim 47 or 48, wherein the method further comprises a step of providing a secondary urging force to move the distal portion at least partially back towards the first position.

50. The method of any one of claims 47 to 49, wherein the method further comprises a further step of constraining the distal portion and the proximal portion along a needle axis as the needle is moves between the first position and the second position.
